(19) United States
(12) Patent Application Publication
(10) Pub. No.: US 2010/0030193 A1
(43) Pub. Date: Feb. 4, 2010

(54) GUIDE CATHETER AND METHOD OF MAKING SAME

(75) Inventor: Brian M. Packard, Monticello, MN (US)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(73) Assignee: Minnesota MedTec, Inc.

(21) Appl. No.: 12/533,908

(22) Filed: Jul. 31, 2009

Related U.S. Application Data
(63) Continuation of application No. 10/818,135, filed on Apr. 5, 2004.
(60) Provisional application No. 60/460,544, filed on Apr. 4, 2003.

(51) Int. Cl. A61M 25/00

(52) U.S. Cl. 604/527; 604/528

(57) ABSTRACT

The present invention is a guide catheter and a method of making the guide catheter. The invention includes a catheter having an inner layer, a support element associated with the inner layer, and an outer layer external to the support element. The invention further includes a cutting apparatus used in the method of making the guide catheter.

### CROSS-REFERENCE TO RELATED APPLICATION(S)

This application claims priority from U.S. provisional application No. 60/460,544, filed Apr. 4, 2003, which is hereby incorporated by reference in its entirety.

### FIELD OF THE INVENTION

The present invention relates to a guide catheter and a method of manufacturing the guide catheter. Further, the present invention relates to a guide catheter configured to be used with expandable devices and devices with sharp components without damage to the catheter, and a method of manufacturing such a catheter.

### BACKGROUND OF THE INVENTION

Guide catheters are typically used to guide instruments such as balloon catheters, guidewires or similar devices to specific locations in a human body to perform their specific function, such as angioplasty. The inner layer of such catheters range from 0.0005 inches to 0.0015 inches. One problem with current guide catheters is that they are damaged or rendered inoperable due to weakness in the materials as a result of the insertion of current expandable nickel titanium devices or devices with sharp components.

There is a need in the art for a guide catheter that has a thin wall thickness yet has the wall strength to withstand the use of expandable devices made of such materials as nickle titanium or devices with sharp components.

### BRIEF SUMMARY OF THE INVENTION

The present invention, in one embodiment, is a guide catheter. The catheter has an inner layer, a support element associated with the inner layer, and an outer layer external to the support element. The inner layer has a thickness of from about 0.0015 inches to about 0.006 inches. The support element is configured to provide shape retention to the guide catheter.

In another embodiment, the present invention is a cutting apparatus. The cutting apparatus has a rotatable base component, at least two cutting blades pivotably attached to the base component, and a positioning element configured to move the cutting blades between a cutting position and a non-cutting position.

The present invention, in a further embodiment, is a method of attaching a tip to a catheter. The method includes cutting the catheter with a rotational cutter, heating material with a heated die, and forming the material into a tip on the catheter.

### BRIEF DESCRIPTION OF THE DRAWINGS

FIG. 1A is a side view of a guide catheter, according to one embodiment of the present invention.

FIG. 1B is a perspective view of a portion of a guide catheter, according to one embodiment of the present invention.

FIG. 1C is a side view of a catheter tip, according to one embodiment of the present invention.

FIG. 2A is a side view of a guide catheter, according to an alternative embodiment of the present invention.

FIG. 2B is a side view of a catheter tip, according to one embodiment of the present invention.

FIG. 3A is a cutaway side view of a connection element, according to one embodiment of the present invention.

FIG. 3B is a cutaway side view of a connection element mated with another connection element, according to one embodiment of the present invention.

FIG. 3C is a cutaway side view of a connection element, according to an alternative embodiment of the present invention.

FIG. 3D is a cutaway side view of a connection element mated with another connection element, according to an alternative embodiment of the present invention.

FIG. 4 is a flow chart depicting a method of manufacturing a catheter, according to one embodiment of the present invention.

FIG. 5A is a top view of a cutting apparatus, according to one embodiment of the present invention.

FIG. 5B is a side view of a cutting apparatus, according to one embodiment of the present invention.

### DETAILED DESCRIPTION

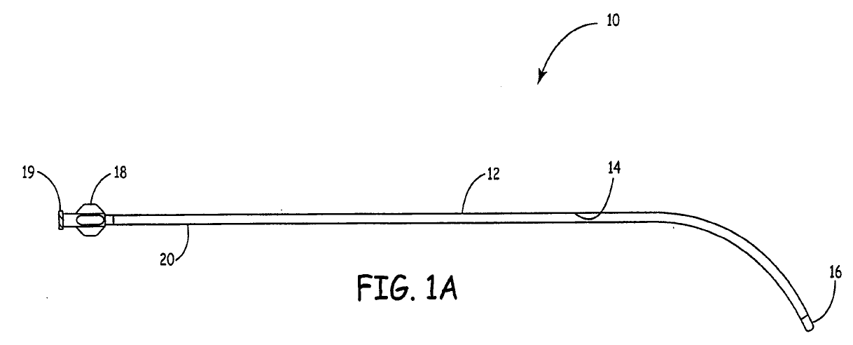

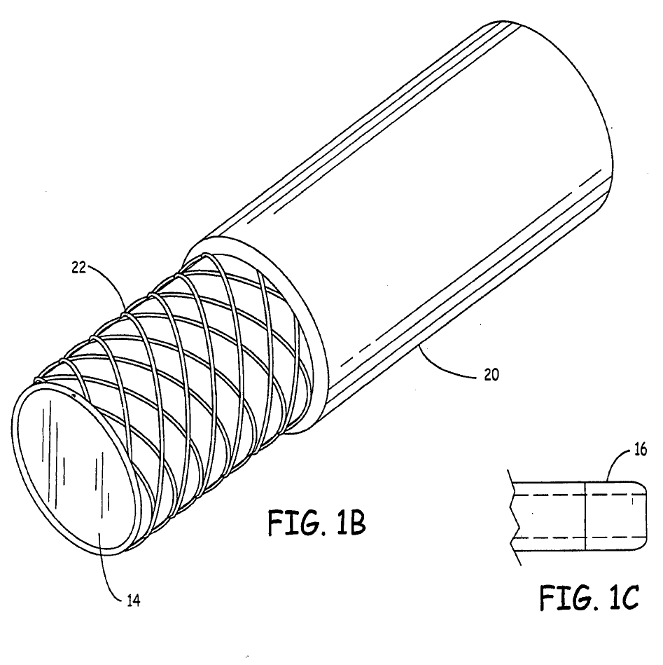

FIG. 1A depicts a guide catheter 10 according to one embodiment of the present invention. The guide catheter 10 has an elongated tubular member 12 having an inner layer 14, a catheter tip 16, a connection element 18 including a male element 19, and an outer layer 20. According to one embodiment, the guide catheter 10 is a sheath guide catheter configured to be used in conjunction with a dilator guide catheter wherein the dilator is inserted into the sheath, as will be described in further detail herein.

The inner layer 14 according to one embodiment has a thickness of from about 0.0015 inches to about 0.006 inches. The layer 14 further may be a slippery inner surface configured to promote the advancement of any device inserted into the guide catheter 10. According to one embodiment, the layer 14 is any fluoropolymer. For example, according to one embodiment the layer 14 is comprised of PTFE. Alternatively, the layer 14 is comprised of MFA. In a further alternative, the inner layer 14 is any similar low-friction material.

The thickness of the inner layer 14 provides a surface that is difficult to damage by insertion of abrasive objects or devices that apply circumferential forces. Further, the thickness of the inner layer 14 prevents the guide catheter 10 from producing unwanted debris and allows for insertion into the body vessels without creating complications.

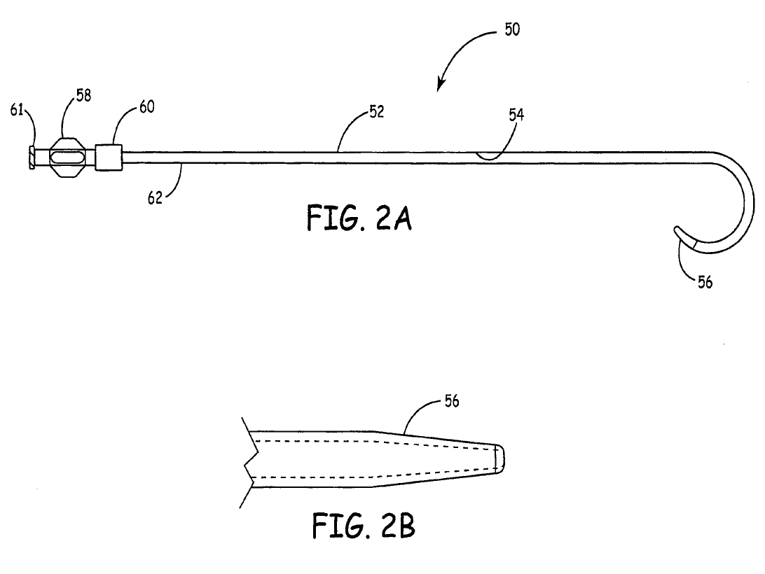

FIG. 1B depicts a portion of a guide catheter 10, according to one embodiment of the present invention. The guide catheter 10 has a support element 22 integrated into the catheter 10. According to one embodiment, the support element 22 is braided wire. Alternatively, the support element 22 is a flexible, kinkless coil. As shown in FIG. 2, the support element 22 is wrapped around an external portion of the inner layer 14. The thickness of the inner layer 14 optimizes the ability to include the support element 22. According to one embodiment, the support element 22 is comprised of tungsten.

The support element 22 is configured to provide a predetermined shape to the guide catheter 10 that can aide the operator in accessing a desired location for use. Further, according to one embodiment, the support element 22 provides a stiffness or rigidity that allows an operator to steer or direct the catheter 10 to difficult locations that require that the catheter 10 withstand resistance. For example, according to one embodiment, the catheter 10 is used to access a membranous ventricle septal defect (“membranous VSD”). According to an alternative aspect of the invention, the support element 22 provides a kink resistance to the catheter 10, such that when the catheter 10 is bent or deformed, no kink or permanent deformation results. For example, according to one embodiment, the support element 22 allows the catheter 10 to be used with a tortuous device such that the tortuous device can be inserted into or through the catheter 10 without resulting in permanent kinks or deformation to the catheter 10.

The outer layer 20 according to one embodiment is configured to be exterior to the support element 22. Further, the outer layer 20 may conform to the shape of the support element 22 and, according to one embodiment in which the support element 22 is braided wire, can be attached to the inner layer 14 in the gaps (also referred to as “pics”) between the braided wires.

The connection element 18 is associated with the tubular member 12 at the proximal end of the tubular member 12. The connection element 18 according to one embodiment is configured to receive devices. According to one embodiment, the connection element 18 has an internal diameter (“I.D.”) that matches the outer diameter (“O.D.”) of the tubular member 12. According to a further embodiment, the connection element 18 has a male element 19 configured to be coupleable with a female element on a connection device or loader. In operation, the insertion into the connection element 18 of a connection device or loader having an O.D. that is the same as the tubular member 12 allows for smooth insertion of a device through the connection device or loader and into the guide catheter 10.

FIG. 1C depicts a catheter tip 16, according to one embodiment of the present invention. The catheter tip 16 is associated with the distal end of the guide catheter 10. The tip 16 is configured to prevent portions of the support element 22 to be exposed at the end of the catheter 10.

FIG. 2A depicts a guide catheter 50, according to an alternative embodiment of the present invention. The guide catheter 50 has an elongated tubular member 52 having an inner layer 54, a catheter tip 56, a connection element 58 including a female element 60 and a male element 61, and an outer layer 62. According to one embodiment, the guide catheter 50 is a dilator guide catheter configured to be used in conjunction with a sheath guide catheter such as, for example, guide catheter 10, wherein the dilator is inserted into the sheath, as will be described in further detail herein.

The inner layer 54 and outer layer 62, according to one embodiment, have the same or substantially the same characteristics, composition, and structure as the inner layer 14 and outer layer 20, respectively, described herein. According to an alternative aspect of the invention, the guide catheter 50 has a support element (not shown) integrated into the catheter 50, wherein the support element has the same or substantially the same characteristics, composition, and structure as the support element 22 described herein. In a further alternative, the guide catheter 50 has no support element.

FIG. 2B depicts a catheter tip 56 according to one embodiment of the present invention. The catheter tip 56 is associated with the distal end of the guide catheter 50. The tip 56 is configured to prevent portions of the support element (not shown) to be exposed at the end of the catheter 50.

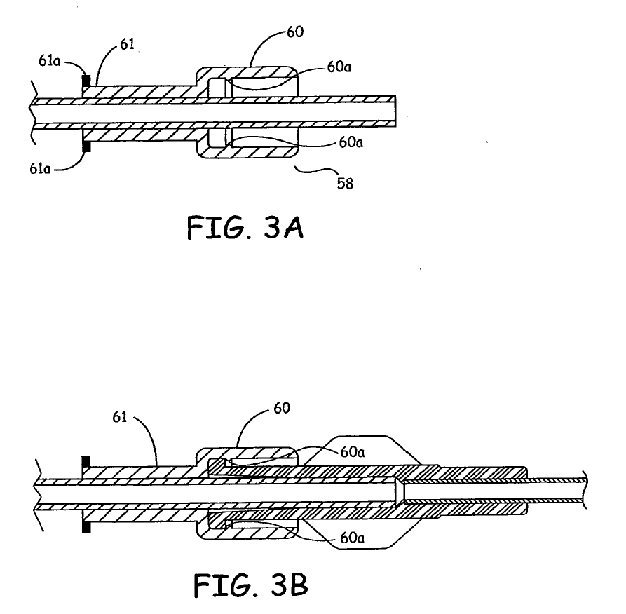

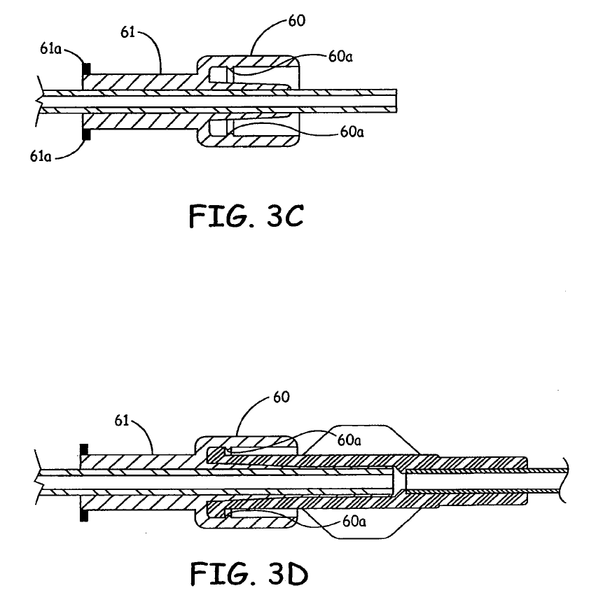

FIG. 3A depicts a connection element 58, according to one embodiment of the present invention. The connection element 58 is associated with the tubular member 52 at the proximal end of the tubular member 52 as shown in FIG. 2A. The connection element 58 according to one embodiment is configured to receive devices and further to connect to devices that the tubular member 52 is inserted into. According to one embodiment, the connection element 58 has an internal diameter (“I.D.”) that matches the outer diameter (“O.D.”) of the tubular member 52. In operation, the insertion into the connection element 58 of a connection device or loader having an O.D. that is the same as the tubular member 52 allows for smooth insertion of a device through the connection device or loader and into the guide catheter 50.

According to a further aspect of the invention, the connection element 58 as depicted in FIG. 3A has a male element 61 configured to be coupleable with a female element on a connection device or loader that is inserted into the catheter 50. The male element 61 has protruding elements 61a that are configured to contact a female element such that the male 61 and female elements are held in connection and can be separated only with some force being applied.

According to another embodiment, the connection element 58 has a female element 60 as shown in FIG. 3A configured to be coupleable with a male element on a device into which the catheter 50 is inserted. The female element 60 has inner protruding elements 60a that are configured to contact protruding elements on a male element (similar to the protruding elements 61a as shown) such that the male and female 60 elements are held in connection and can be separated only with some force being applied, as shown in FIG. 3B.

FIG. 3C depicts a connection element 58, according to an alternative embodiment of the present invention. The connection element 58 has a female element 60 with inner protruding elements 60a and a male element 61 with protruding elements 61a. FIG. 3D shows the female element 60 of FIG. 3C in connection with a male element.

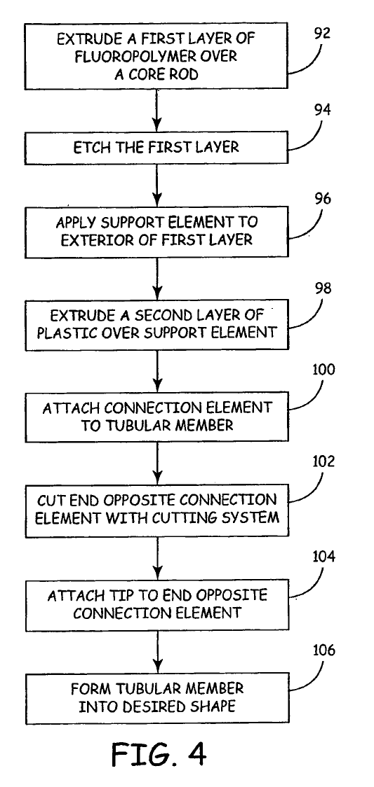

FIG. 4 depicts a method of manufacturing a catheter 90, according to one embodiment of the present invention. According to one embodiment, the method is a method of manufacturing a sheath guide catheter. Alternatively, the method is a method of manufacturing a dilator guide catheter. First, a first layer of a fluoropolymer is extruded onto a core rod (block 92). In an alternative aspect of the invention, the extruded material can be any known extrudable polymer. According to one embodiment the core rod is copper. A copper rod can be stretched after the fluoropolymer has been extruded onto, thereby causing the diameter of the rod to decrease and simplifying the removal of the rod from the formed first layer. Alternatively, the core rod is plastic. According to one embodiment, this first layer will be the inner layer 14, 54 of the catheter 10, 50.

The extruded layer is then etched to create a surface to which other objects can be attached (block 94). According to one embodiment, the etching takes place by applying a sodium-based solution to the layer. In an alternative aspect of the invention, the extruded layer is not etched. Next, according to one embodiment, the support element 22 is applied to the exterior of the layer (block 96). According to one embodiment, the support element 22 is applied by braiding the layer with wires. The layer may be braided with from about 8 to about 32 wires. Alternatively, the support element 22 is a kink-resistant flexible coil that is applied to the exterior of the layer. In an alternative embodiment, no support element is applied. For example, the manufacture of some dilator guide catheters does not require application of a support element.

A second layer of plastic is then extruded over the support element 22 (block 98), or if there is no support layer, the second layer is extruded over the first layer. According to one embodiment, the plastic is nylon. Alternatively, the plastic can be any known plastic for use in medical devices. According to one embodiment, air pressure is applied during this step to ensure that the second layer extends through gaps in the support element 22 and attaches to the first layer.

Once the tubular member has been manufactured, additional components can be added to create the catheter. The connection element 18, 58 is attached to an end of the tubular member 12, 52 (block 100). According to one embodiment, the connection element 18, 58 is attached by molding the connection element 18, 58 onto the end of the tubular member 12, 52. That is, an appropriate mold is placed on the end of the tubular member 12, 52 and hot liquid material is added to the mold such that the material forms a connection element 18, 58 that is molded to the end of the tubular member 12, 52. According to one embodiment, the molding step is accomplished with a molding machine. Alternatively, the connection element 18, 58 is attached by any known means for attaching a component to a catheter.

In one alternative embodiment, the end of the tubular member 12, 52 is cut with a cutting system (block 102) prior to attachment of a tip 16, 56. For some embodiments, cutting the end serves to expose an end of the tubular member and facilitate attachment of a tip. In a further alternative, cutting the end of a tubular member having a support member exposes the support member as well, thereby facilitating complete encapsulation of the support member with the tip. According to one embodiment, a mandrel is inserted into the tubular member 12, 52 prior to the cutting step to facilitate cutting by providing support to the tubular member 12, 52 during the process. In a further embodiment, the cutting system used is a two-blade cutting system described in further detail below.

A tip 16, 56 is then attached to the end of the tubular member 12, 52 opposite the connection element 18, 58 (block 104). According to one embodiment, the tip is formed from an existing portion of the end of the tubular member 12, 52. The end is heated by the application of radio frequency (“R.F.”) energy and then shaped appropriately. Alternatively, the tip is formed by a molding step in which an appropriate piece of plastic is heated, molded into the appropriate shape, and formed onto the catheter using R.F. energy. According to one embodiment, the R.F. energy is applied using R.F. dies.

In accordance with one alternative aspect of the present invention, the resulting catheter 10, 50 is then formed into a desired shape. That is, the catheter 10, 50 is placed in hot liquid to make the catheter moldable. Alternatively, the catheter 10, 50 may be placed on heated platens to make it moldable. The catheter 10, 50 can then be formed into the desired shape. Subsequently, the catheter 10, 50 is placed in cold liquid to eliminate its moldability.

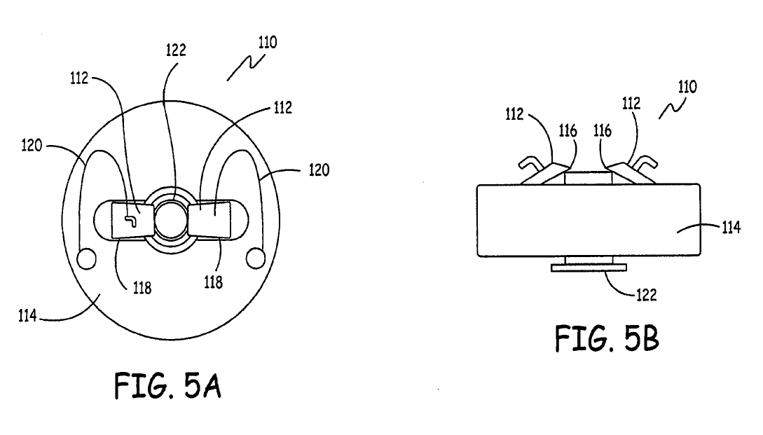

FIG. 5A depicts a top view of a cutting system 110, according to one embodiment of the invention. FIG. 5B depicts a side view of a cutting system 110, according to one embodiment of the invention. According to one embodiment, the cutting system 110 can be used to cut the tubular member 12, 52 as described above. The cutting system 110 has two blades 112 with cutting edges 116. The blades 112 are pivotably coupled to a base 114 with pivot rods 118 inserted through holes at the non-cutting end of the blades. The cutting system 110 also has tension wires 120 connected at one end to the base 114 and at the other end to the blades 112. The tension wires 120 provide a tension urging the cutting edges 116 of the blades 112 toward the base 114. Alternatively, the cutting system 110 can have any known component configured to urge the blades 112 toward the base 114 or provide a downward force or tension on the blades 112 toward the base 114.

The system 110 has a positioning element 122 moveably disposed in the center of the base 114 and in contact with both blades 112. According to one embodiment, the positioning element 122 is a tube element. The tube element 122 is configured to move the blades 112 between a cutting position and a non-cutting position. That is, when the tube 122 is urged upward (toward the blades side of the base 114), the blades 112 are urged upwards and the distance between the cutting edges 116 increases. When the upward force on the tube 122 is removed, the downward force of the tension wires 120 urges the blades 112 downward and the distance between the cutting edges 116 decreases. Alternatively, the positioning element 122 can be any component configured to move the blades 112 between a non-cutting position and a cutting position. The base 114 is configured to rotate or spin around the tube element 122 such that a tubular member 12, 52 disposed within the tube element 122 can be cut by the two blades 112.

According to one embodiment, the two blades 112 cut at two different locations around the circumference of the tubular member 12, 52, thus applying an equal amount of pressure around the circumference and cutting in a precise manner that prevents exposure of any portion of the support element 22 by forcing the support element 22 inward as it cuts. Alternatively, the cutting system 110 can have three blades 112. In a further alternative, the cutting system 110 can have 1 to 4 blades 112.

In operation, the cutting system 110 can be used to cut a tubular member 10, 50. First, the tube element 122 is urged upward, thereby urging the blades 112 upward and increasing the distance between them. When the tube element 122 has urged the blades 112 upward such that the distance between the blades 112 is greater than the O.D. of the tubular member 12, 52 to be cut, the tubular member 12, 52 is inserted through the tube element 122. Once the tubular member 12, 52 is properly positioned, the force on the tube element 122 is released and the blades 112 are urged downward and closer together by the tension wires 120 until they are in contact with the tubular member 12, 52. Then the base 114 is caused to rotate or spin such that the blades 112 spin around the tubular member 12, 52, thereby cutting the tubular member 12, 52.

While multiple embodiments are disclosed, still other embodiments of the present invention will become apparent to those skilled in the art from the following detailed description, which shows and describes illustrative embodiments of the invention. As will be realized, the invention is capable of modifications in various obvious aspects, all without departing from the spirit and scope of the present invention. Accordingly, the drawings and detailed description are to be regarded as illustrative in nature and not restrictive.

Although the present invention has been described with reference to preferred embodiments, persons skilled in the art will recognize that changes may be made in form and detail without departing from the spirit and scope of the invention.

1-8. (canceled)

9. A guide catheter for use with an introducer to treat a membranous septal defect, the introducer having an outer diameter, the guide catheter comprising:
a catheter portion including:
an inner layer with an etched outer surface;
a support element attached to the etched outer surface, the support element configured to provide shape retention to the guide catheter; and
a pressure applied outer layer external to the support element, the outer layer defining an outer diameter; wherein
an inner diameter of the guide catheter is adapted to receive and pass through an expandable device for the treatment of septal defects; and
a connection portion positioned over a proximal end of the catheter portion, the connection portion comprising a tubular shaft with an inner and an outer surface, the inner surface defined by a first inner diameter and a second inner diameter with a transition there between, the first inner diameter being substantially equal to the outer diameter of the catheter portion and the second inner diameter being substantially equal to the outer diameter of the introducer, the proximal end of the catheter portion being positioned adjacent to the transition, and the connection portion further comprising a circumferential protruding rib positioned on the outer surface of the tubular shaft adjacent the proximal end of the connection portion, the rib being adapted to engage a corresponding rib on the introducer to form a positive connection.

10. The guide catheter of claim 9 wherein the support element is a braided wire.

11. The guide catheter of claim 9 wherein the support element is a kinkless coil.

12. The guide catheter of claim 9 wherein the pressure applied outer layer attaches to the inner layer through the gaps in the support element.

13. The guide catheter of claim 9 wherein the support element is made of tungsten.

14. The guide catheter of claim 9 wherein the inner layer has a thickness of from about 0.0015 inches to about 0.006 inches.

15. A guide catheter for use with an introducer to treat a membranous septal defect, the introducer having an outer diameter, the guide catheter comprising:
a catheter portion including:
an inner layer with an etched outer surface;
a support element attached to the etched outer surface, the support element configured to provide shape retention to the guide catheter; and
a pressure applied outer layer external to the support element, the outer layer defining an outer diameter; wherein
an inner diameter of the guide catheter is adapted to receive and pass through an expandable device for the treatment of septal defects; and
a connection portion positioned over a proximal end of the catheter portion, the connection portion comprising a tubular shaft with an inner and an outer surface, the inner surface defined by a first inner diameter and a second inner diameter with a transition there between, the first inner diameter being substantially equal to the outer diameter of the catheter portion and the second inner diameter being substantially equal to the outer diameter of the introducer, the proximal end of the catheter portion being positioned adjacent to the transition, and the connection portion further comprising an endless circumferential protruding rib positioned on the outer surface of the tubular shaft adjacent the proximal end of the connection portion, the rib being adapted to engage a corresponding endless circumferential protruding rib on the introducer to form a positive connection.

16. A method of using a guide catheter to treat a membranous septal defect comprising:
inserting the guide catheter into a body vessel;
steering the guide catheter to a membranous septal defect;
inserting a device into a loader;
attaching the loader to the guide catheter; and
further inserting the device into the guide catheter to access the membranous septal defect.
